(19) United States
(12) Patent Application Publication
(10) Pub. No.: US 2025/0376695 A1
(43) Pub. Date: Dec. 11, 2025

(54) AAV-BASED GENE THERAPY FOR HEREDITARY ANTITHROMBIN DEFICIENCY

(71) Applicant: UNION HOSPITAL, TONGJI MEDICAL COLLEGE, HUAZHONG UNIVERSITY OF SCIENCE AND TECHNOLOGY, Wuhan (CN)

(72) Inventors: Yu Hu, Wuhan (CN); Liang Tang, Wuhan (CN); Yanyi Tao, Wuhan (CN); Hui Lu, Wuhan (CN)

(21) Appl. No.: 19/190,333

(22) Filed: Apr. 25, 2025

(30) Foreign Application Priority Data
May 24, 2024 (CN) 202410654767.X

(51) Int. Cl. C12N 15/86; A61K 48/00; A61P 7/02

(52) U.S. Cl. CPC C12N 15/86; A61K 48/0058; A61P 7/02; C12N 2750/14122; C12N 2750/14143; C12N 2830/008; C12N 2830/50

(57) ABSTRACT

Disclosed is an AAV-based gene therapy for hereditary antithrombin deficiency, which belongs to the technical field of gene therapy. According to the present disclosure, the long-term efficacy and safety of AAV vector-mediated hSERPINC1 gene delivery in the treatment of hereditary AT deficiency is explored for the first time, demonstrating its efficacy in restoring the level and activity of AT antigen in the plasma of AT-deficient mice, rebalancing the coagulation and anticoagulation systems in the body, and reducing the thrombotic tendency of mice. AAV gene therapy is expected to achieve permanent cure of AT deficiency. This discovery provides a preclinical basis for the clinical transformation of AAV therapy for the treatment of AT deficiency, and provides a new treatment direction for the long-term radical cure of patients with thrombophilias such as hereditary antithrombin deficiency.

### CROSS-REFERENCE TO RELATED APPLICATION

This application claims priority of Chinese Patent Application No. 202410654767.X, filed on May 24, 2024, the entire contents of which are incorporated herein by reference.

### REFERENCE TO A SEQUENCE LISTING SUBMITTED VIA EFS-WEB

The content of the xml file of the sequence listing named “HKIP-US-1-1312-07-sequence_listing-ST26” which is 6,838 b in size was created on Apr. 8, 2025 and electronically submitted via EFS_Web herewith. These sequence listing is incorporated herein by reference in its entirety.

### TECHNICAL FIELD

The present disclosure belongs to the technical field of gene therapy, and in particular relates to an AAV-based gene therapy for hereditary antithrombin deficiency.

### BACKGROUND

Hereditary antithrombin (AT) deficiency is an autosomal dominant disease with a prevalence of approximately 0.02%-0.2% in the general population and approximately 1%-5% in patients with venous thromboembolism (VTE). Among the known inherited thrombotic susceptibility disorders, AT deficiency carries a high risk of VTE. Patients with AT deficiency have an increased risk of first VTE (hazard ratio: 14.0; 95% CI, 5.5-29.0), and the annual risk of VTE recurrence is 8.8% (95% CI, 4.6-14.1). In addition, AT deficiency is one of the most common causes of thromboembolic events in children. The main clinical manifestation of AT heterozygous loss is venous thrombosis, and some patients may also experience arterial thrombotic events such as myocardial infarction. Homozygous deletion of AT usually leads to embryonic death, or giant thrombosis, purpura fulminans and disseminated intravascular coagulation in newborns, making it difficult for them to survive.

Considering the high incidence, recurrence rate and severity of early thromboembolic events in patients with AT deficiency, it is clinically recommended that symptomatic AT mutation gene carriers and asymptomatic AT mutation gene carriers with other high-risk factors for thrombosis use drugs such as vitamin K antagonists (VKA) for lifelong anticoagulation therapy. However, some patients with severe AT deficiency still experience recurrent thrombosis despite adequate anticoagulation. The direct oral anticoagulants currently widely used in clinical practice have certain limitations, and their preventive effect on thromboembolic events is limited in patients with severe thrombophilia. In addition, patients with AT deficiency who receive long-term anticoagulation therapy require continuous clinical monitoring and monitoring of related laboratory parameters, as such patients show resistance to heparin when receiving anticoagulation therapy and may be at risk of death from massive bleeding.

In order to solve the treatment dilemma of patients with AT deficiency in clinical practice and achieve a safe and effective long-term radical cure, we consider the use of gene therapy. The applicant has previously successfully used CRISPR-CAS9 gene editing technology to correct the SERPINC1 mutation in induced pluripotent stem cells (iPSCs) derived from patients with severe homozygous AT deficiency. Injecting these iPCS-differentiated hepatocytes into the spleen of AT-deficient mice successfully normalized the plasma AT concentration and reduced thrombosis in mice. The study has been published in Science. This study shows that gene therapy is a promising research direction for curing AT deficiency. The disadvantage is that gene editing methods need to be customized according to different locations and types of gene mutations and cannot be universal. The supplementary treatment methods such as normal gene delivery can overcome this shortcoming and are applicable to various types of AT-deficient gene mutations.

AAV has attracted extensive attention and application due to its advantages in effectively, continuously and safely expressing exogenous genes in low-immunogenic host cells, and presently it has become the preferred gene therapy vector and the most promising gene therapy tool. The rAAV vectors have entered Phase II-III clinical trials for the treatment of hemophilia, but there are no relevant research reports on their use in the treatment of hereditary thrombophilia.

### SUMMARY

An object of the present disclosure is to provide a recombinant adeno-associated virus (rAAV) virion, wherein the rAAV virion includes an AAV8 serotype capsid, an AAV2 type gene skeleton, and a gene encoding SRPINC1;
a vector genome includes, in a 5′ to 3′ direction:
(1) a TBGp promoter;
(2) a SERPINC1 gene shown in SEQ ID NO.1;
(3) a SV40 PolyA;
there is an arbitrary linker between (1) and (2), and between (2) and (3).

Preferably, the vector genome includes, in a 5′ to 3′ direction:
(1) a TBGp promoter;
(2) a SERPINC1 gene shown in SEQ ID NO.1;
(3) a Linker shown in SEQ ID NO.2;
(4) a Luc fluorescent tag shown in SEQ ID NO.3;
(5) a SV40 PolyA;
there is an arbitrary linker between (1) and (2), and between (4) and (5).

Preferably, the AAV8 serotype capsid includes AAV8 VP1, AAV8 VP2 and AAV8 VP3.

Another object of the present disclosure is to provide a pharmaceutical composition, and the pharmaceutical composition includes the aforementioned rAAV virion.

Preferably, the pharmaceutical composition further includes a pharmaceutically acceptable excipient.

More preferably, the rAAV virion is the only active ingredient in the pharmaceutical composition.

Still another object of the present disclosure is to provide use of the rAAV virion or the pharmaceutical composition in the preparation of a product for treating hereditary antithrombin deficiency.

Compared with the prior art, the present disclosure can achieve the following beneficial effects.

The long-term efficacy and safety of AAV vector-mediated hSERPINC1 gene delivery in the treatment of hereditary AT deficiency is explored for the first time, demonstrating its efficacy in restoring the level and activity of AT antigen in the plasma of AT-deficient mice, rebalancing the coagulation and anticoagulation systems in the body, and reducing the thrombotic tendency of mice. AAV gene therapy is expected to achieve permanent cure of AT deficiency. This discovery provides a preclinical basis for the clinical transformation of AAV therapy for the treatment of AT deficiency, and provides a new treatment direction for the long-term radical cure of patients with thrombophilias such as hereditary antithrombin deficiency.

### BRIEF DESCRIPTION OF THE DRAWINGS

FIG. 1 is an AAV vector in Example 1, including NM_000488-Linker-Luc, GV632: TBGp-MCS-SV40PolyA, and the order of elements is: TBGp-SERPINC1 (NM_000488-Linker-Luc))-SV40 PolyA.

FIG. 2 is a flow chart of the experiment in Example 1.

FIGS. 3A to 3D show the results of in vivo imaging and the detection results of plasma AT activity and concentration at different time points after the injection of AAV-hSERPINC1 in mice in Example 1, wherein: FIG. 3A is the results of in vivo imaging of mice; FIG. 3B is the results of fluorescence quantitative analysis of in vivo imaging of mice; FIG. 3C is the results of AT activity detection; FIG. 3D is the results of AT antigen level detection.

FIG. 4 is the results of immunofluorescence staining of liver tissue sections of mice at 8 weeks after the injection of AAV-hSERPINC1 in Example 1. The red fluorescence represents AT, and the blue fluorescence represents DAPI.

FIGS. 5A to 5C show the test result of thrombosis tendency of mice 8 weeks after the injection of AAV-hSERPINC1 in Example 1, wherein FIG. 5A is the results of in vitro thrombin generation assay of mouse plasma; FIG. 5B is the results of thrombus weight formed after the deep vena cava stenosis model is constructed in mice; FIG. 5C is the results of thrombus length formed after the deep vena cava stenosis model is constructed in mice.

FIG. 6A, FIG. 6B, FIG. 6C, FIG. 6D, FIG. 6E, FIG. 6F, FIG. 6G and FIG. 6H are the test result of liver, kidney and heart indicators at different time points after the injection of AAV-hSERPINC1 in Example 1.

### DETAILED DESCRIPTION

### Example 1

### 1. Construction of AAV Vector

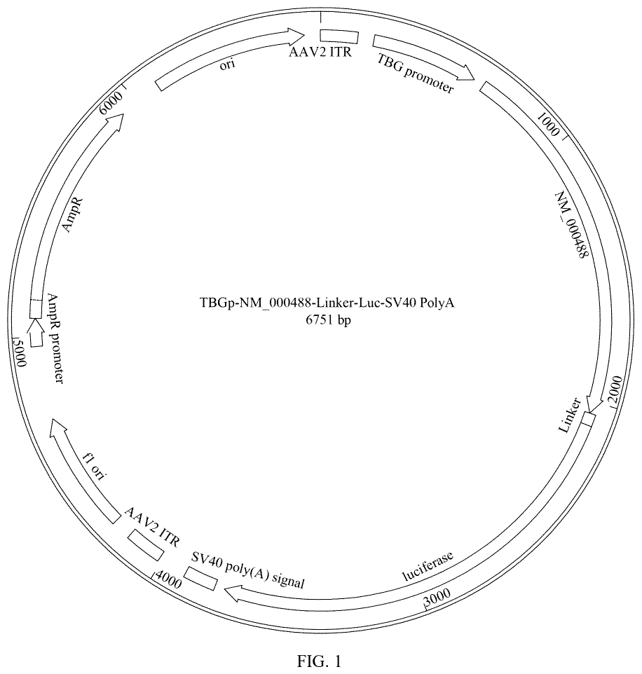

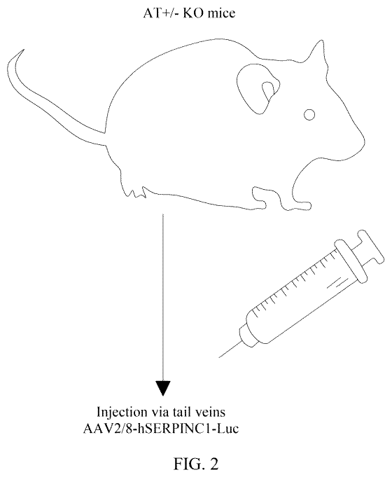

(1) An AAV2/8 chimeric vector (type 2 gene skeleton, type 8 serotype) with liver-specific promoter TBG, hSERPINC1 target gene and Luc fluorescent label was designed and constructed, as shown in FIG. 1.

### (2) NM_000488-Linker-Luc Sequence

(SEQ ID No. 1)
ATGTATTCCAATGTGATAGGAACTGTAACCTCTGGAAAAAGGAAGGTTTATC
TTTTGTCCTTGCTGCTCATTGGCTTCTGGGACTGCGTGACCTGTCACGGGAGCCCTGT
GGACATCTGCACAGCCAAGCCGCGGGACATTCCCATGAATCCCATGTGCATTTACCG
CTCCCCGGAGAAGAAGGCAACTGAGGATGAGGGCTCAGAACAGAAGATCCCGGAG
GCCACCAACCGGCGTGTCTGGGAACTGTCCAAGGCCAATTCCCGCTTTGCTACCACT
TTCTATCAGCACCTGGCAGATTCCAAGAATGACAATGATAACATTTTCCTGTCACCC
CTGAGTATCTCCACGGCTTTTGCTATGACCAAGCTGGGTGCCTGTAATGACACCCTC
CAGCAACTGATGGAGGTATTTAAGTTTGACACCATATCTGAGAAAACATCTGATCAG
ATCCACTTCTTCTTTGCCAAACTGAACTGCCGACTCTATCGAAAAGCCAACAAATCC
TCCAAGTTAGTATCAGCCAATCGCCTTTTTGGAGACAAATCCCTTACCTTCAATGAG
ACCTACCAGGACATCAGTGAGTTGGTATATGGAGCCAAGCTCCAGCCCCTGGACTTC
AAGGAAAATGCAGAGCAATCCAGAGCGGCCATCAACAAATGGGTGTCCAATAAGAC
CGAAGGCCGAATCACCGATGTCATTCCCTCGGAAGCCATCAATGAGCTCACTGTTCT
GGTGCTGGTTAACACCATTTACTTCAAGGGCCTGTGGAAGTCAAAGTTCAGCCCTGA
GAACACAAGGAAGGAACTGTTCTACAAGGCTGATGGAGAGTCGTGTTCAGCATCTA
TGATGTACCAGGAAGGCAAGTTCCGTTATCGGCGCGTGGCTGAAGGCACCCAGGTG
CTTGAGTTGCCCTTCAAAGGTGATGACATCACCATGGTCCTCATCTTGCCCAAGCCT
GAGAAGAGCCTGGCCAAGGTAGAGAAGGAACTCACCCCAGAGGTGCTGCAAGAGT
GGCTGGATGAATTGGAGGAGATGATGCTGGTGGTCCACATGCCCCGCTTCCGCATTG
AGGACGGCTTCAGTTTGAAGGAGCAGCTGCAAGACATGGGCCTTGTCGATCTGTTCA
GCCCTGAAAAGTCCAAACTCCCAGGTATTGTTGCAGAAGGCCGAGATGACCTCTATG
TCTCAGATGCATTCCATAAGGCATTTCTTGAGGTAAATGAAGAAGGCAGTGAAGCA
GCTGCAAGTACCGCTGTTGTGATTGCTGGCCGTTCGCTAAACCCCAACAGGGTGACT
TTCAAGGCCAACAGGCCTTTCCTGGTTTTTATAAGAGAAGTTCCTCTGAACACTATT
ATCTTCATGGGCAGAGTAGCCAACCCTTGTGTTAAG
(SEQ ID No. 2)
GGTGGCGGTGGCTC GGGCGGTGGTGGGTCGGGTGGCGGCGGATCT
(SEQ ID No. 3)

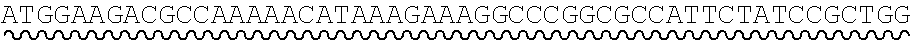

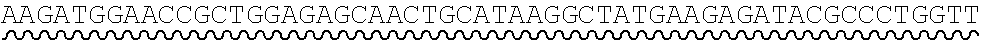

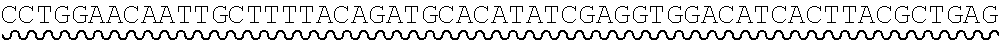

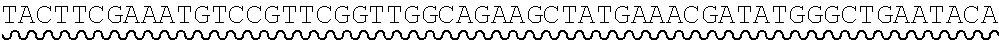

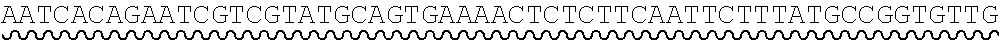

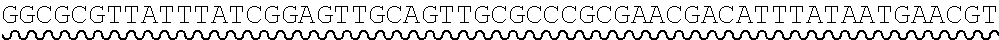

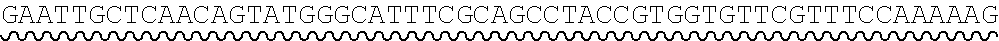

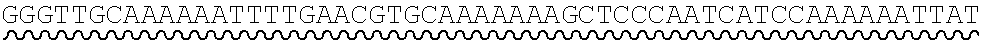

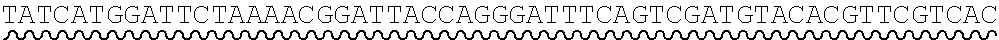

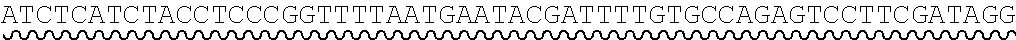

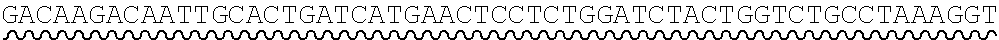

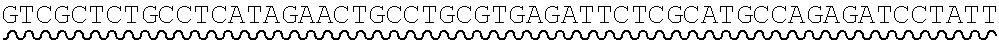

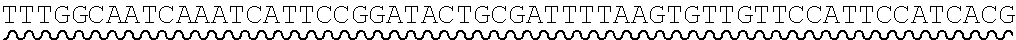

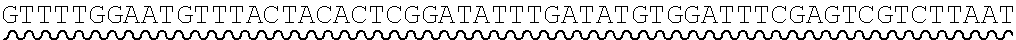

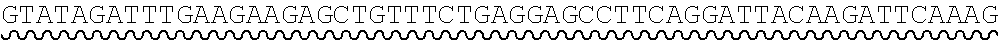

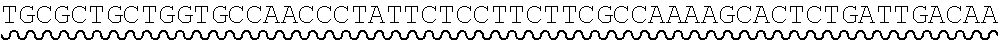

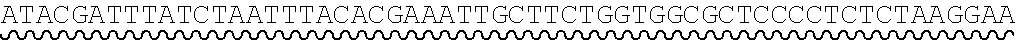

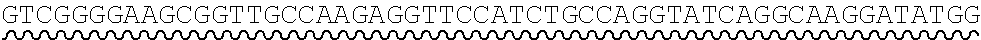

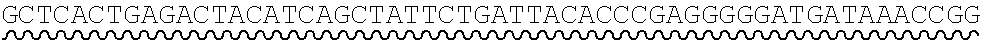

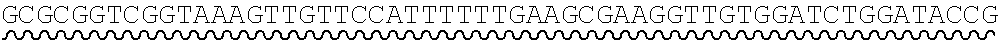

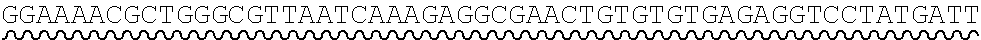

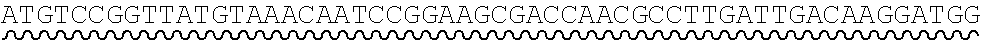

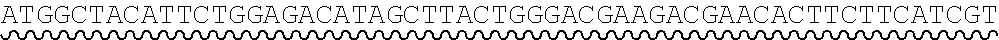

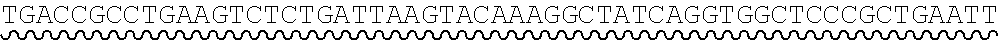

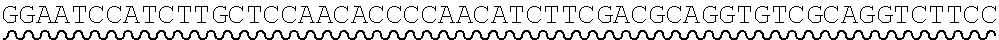

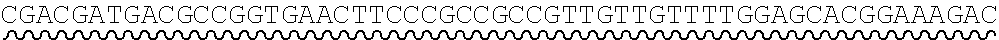

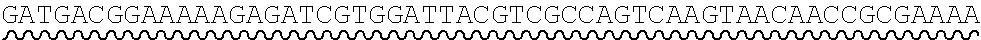

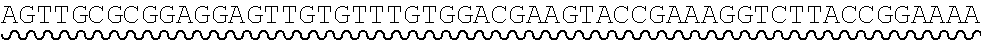

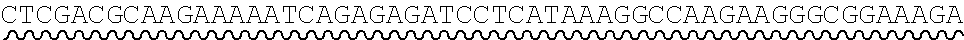

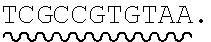

The horizontal line part SEQ ID NO.2 represented the linker sequence, and the wavy line part SEQ ID NO.3 represented the Luc sequence.

### 2. Construction of Knockout Gene Mice

AT+/−KO mice (GenBank Accession Number of the mouse SERPINC1 gene: NM_080844.4) were constructed using CRISPR/Cas9 genome editing tools.

### 3. Experimental Scheme

The liver-specific AAV-hSERPINC1 with Luc tags was injected into 6-week-old AT+/−KO mice via the tail veins in 2-fold gradients and at 3 doses (0.5×10\hat{ }11 vg/mouse; 1×10\hat{ }11 vg/mouse; 2×10\hat{ }11 vg/mouse). Each mouse was subjected to in vivo imaging at different time points to visualize the expression of the vector in the mouse. At the same time, blood was collected through the medial canthus of the eye at this time point, and the AT antigen level and activity of the mouse plasma were measured using the antigen and activity kits. The observation period was as long as 11 months to evaluate whether the thrombotic tendency of AT-deficient mice was improved after AAV gene therapy.

### 4. Experimental Result

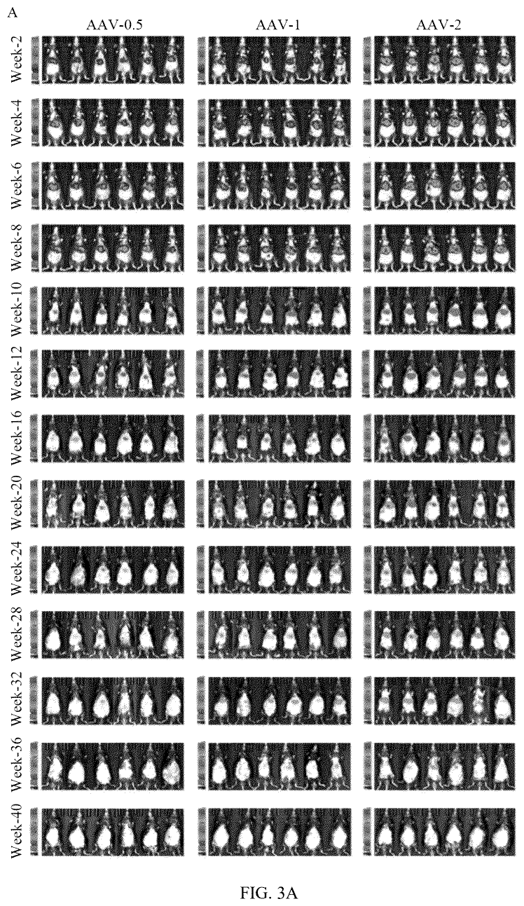

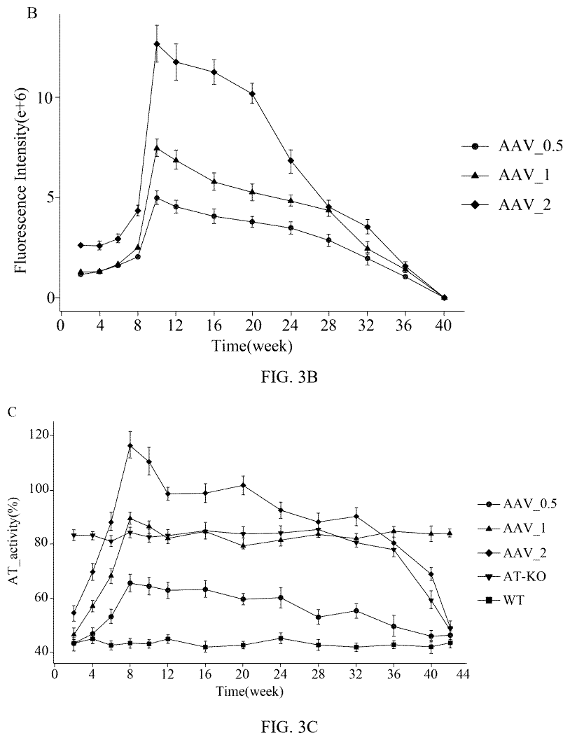

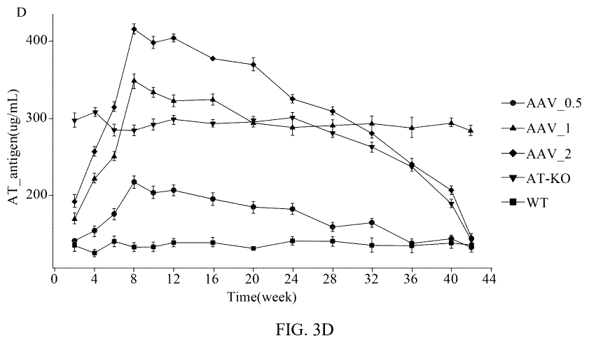

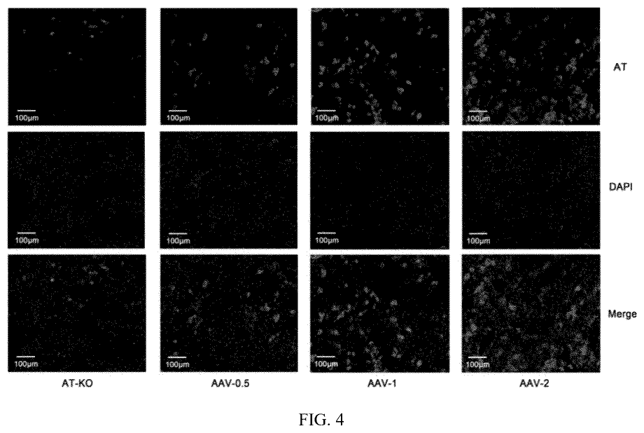

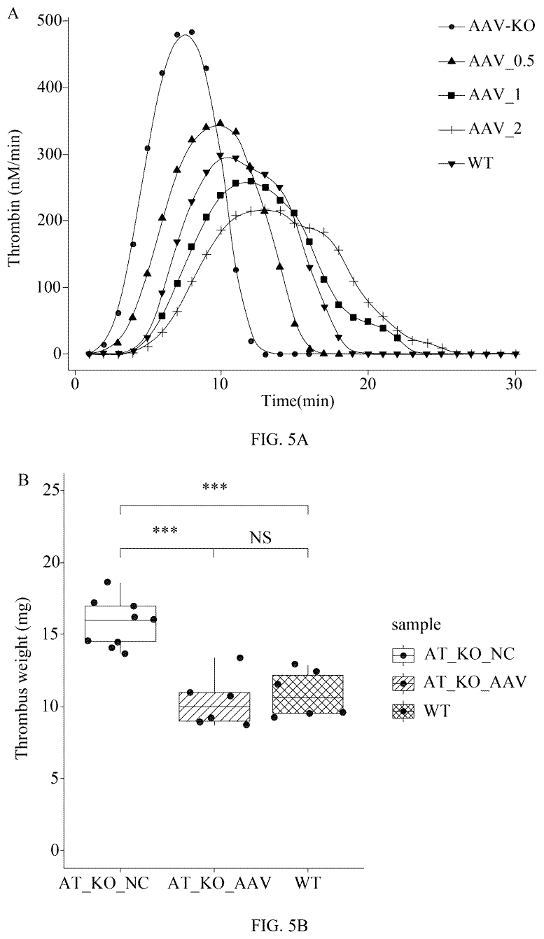

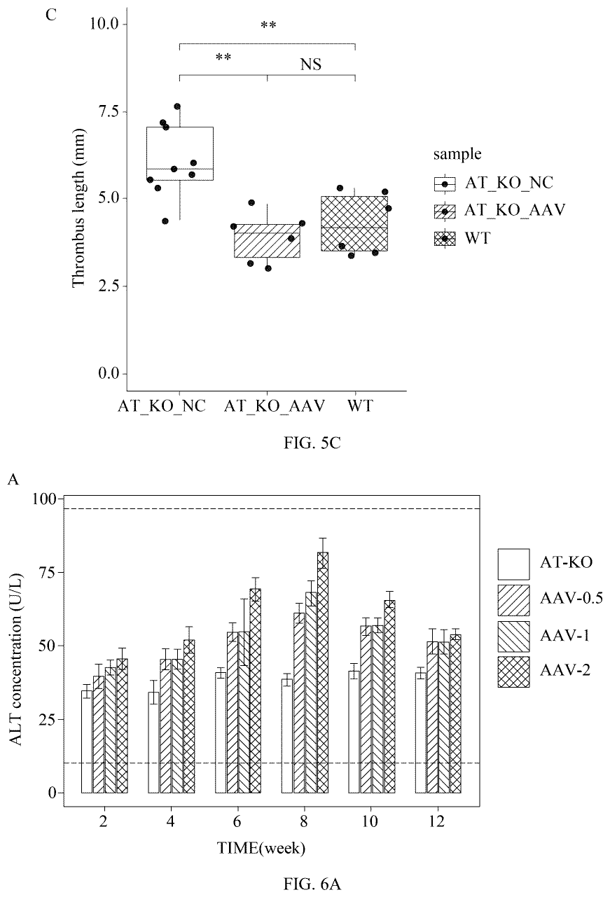

(1) Compared with untreated knockout mice, knockout mice injected with a moderate dose of AAV (1×10\hat{ }11 vg/mouse) maintained plasma AT concentration and activity at the therapeutic levels (activity: increased from 43.3±3.9% to 89.5±5.3%, p<0.0001; concentration: increased from 133.6±12.5 ρg/mL to 347.8±19.6 ρg/mL, p<0.0001) and lasted for up to 40 weeks (FIGS. 3A to 3D). Tissue immunofluorescence results showed that AAV could significantly improve the liver AT level in AT/−KO mice. This indicated that AAV2/8-hSERPINC1 could successfully target the liver and restore the liver's ability to produce AT (FIG. 4).

(2) The mouse inferior vena cava stenosis model showed that the weight of thrombus formed in knockout mice after treatment with AAV at a medium dose was significantly lower than that in the untreated knockout mice (10.32±1.79 mg VS 17.02±4.52 mg, P<0.001), but was not significantly different from that in the wild-type mice (10.88±1.61 mg) (FIGS. 5A to 5C).

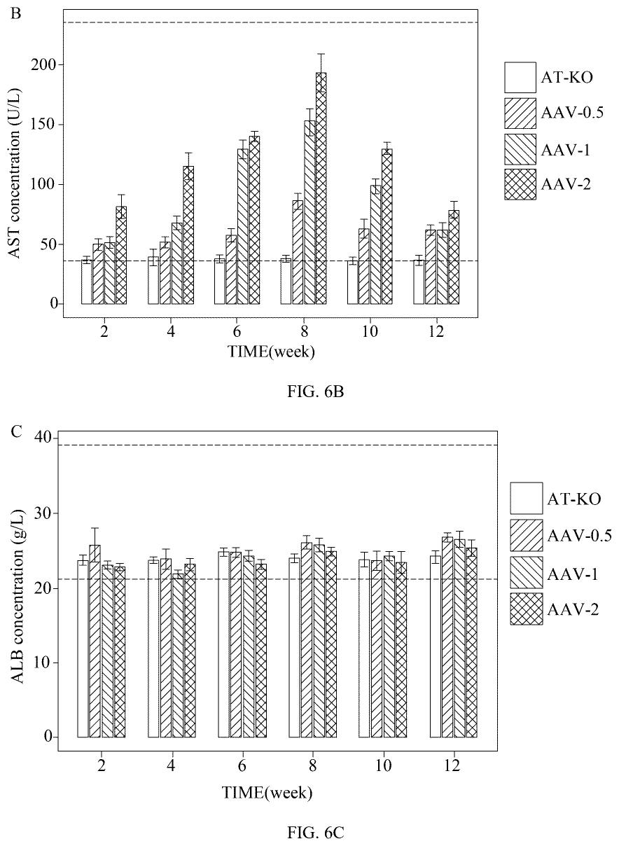

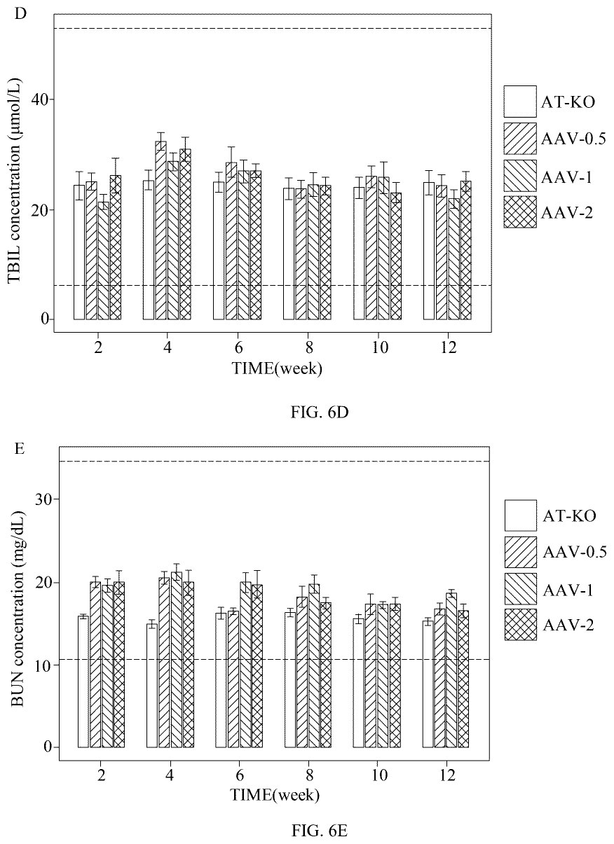

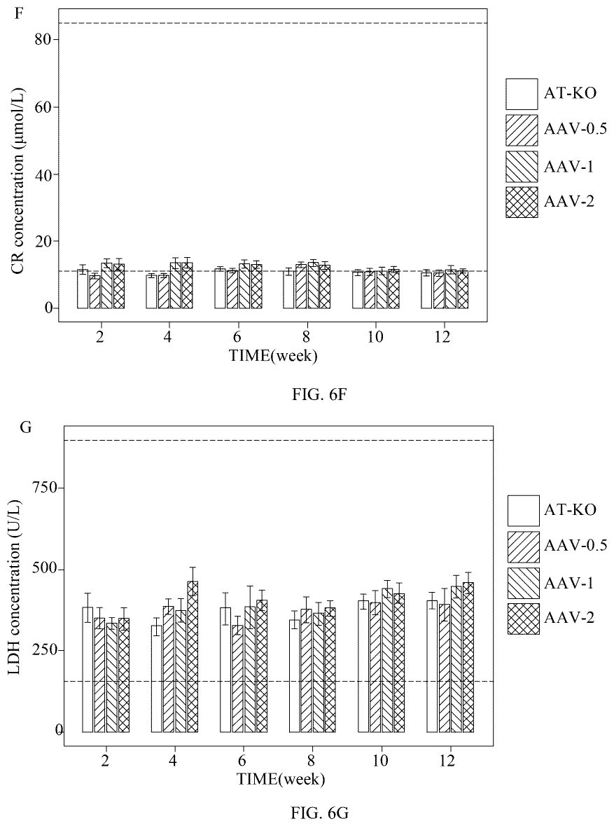

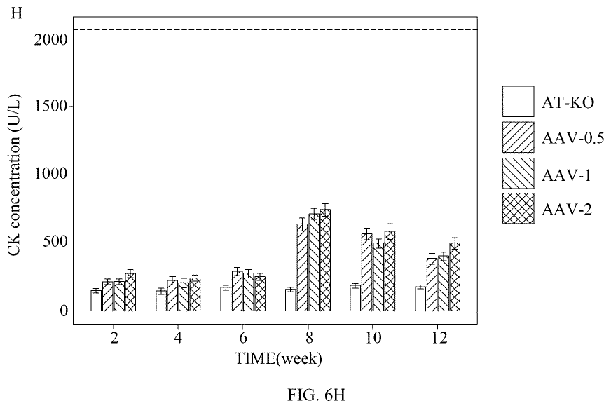

(3) After treatment with AAV, the liver and kidney indicators of mice were tested, and the results showed that the liver enzymes of mice increased in a short period of time and reached the peak in the 8^{th} week, and then gradually declined (FIG. 6). During the AAV treatment observation period, no abnormal phenomena such as death or bleeding occurred in mice in all experimental groups.

### 5. Conclusion

The study showed that adeno-associated virus AAV2/8-hSERPINC1-mediated gene therapy could continuously correct the hypercoagulable state and thrombosis tendency of mice with hereditary antithrombin deficiency. The therapy has been proven to be safe and effective, providing a potential application for radical cure of hereditary antithrombin deficiency and providing a preclinical basis for the clinical transformation of AAV therapy for the treatment of AT deficiency.

The foregoing description merely describes the preferred embodiments of the present disclosure and is not intended to limit the scope of the present disclosure. It should be noted that for those of ordinary skill in the art, several improvements and modifications can be made without departing from the design principle of the present disclosure, and these improvements and modifications shall fall within the scope of protection defined by the appended claims herein.

1. A recombinant adeno-associated virus (rAAV) virion, wherein the rAAV virion comprises an AAV8 serotype capsid, an AAV2 type gene skeleton, and a gene encoding SRPINC1;
wherein a vector genome comprises, in a 5′ to 3′ direction:
(1) a TBGp promoter;
(2) a SERPINC1 gene shown in SEQ ID NO.1;
(3) a SV40 PolyA;
there is an arbitrary linker between (1) and (2), and between (2) and (3).

2. The rAAV virion according to claim 1, wherein the vector genome comprises, in a 5′ to 3′ direction:
(3) a Linker shown in SEQ ID NO.2;
(4) a Luc fluorescent tag shown in SEQ ID NO.3;
(5) a SV40 PolyA;
there is an arbitrary linker between (1) and (2), and between (4) and (5).

3. The rAAV virion according to claim 1, wherein the AAV8 serotype capsid comprises AAV8 VP1, AAV8 VP2 and AAV8 VP3.

4. The rAAV virion according to claim 2, wherein the AAV8 serotype capsid comprises AAV8 VP1, AAV8 VP2 and AAV8 VP3.

5. A pharmaceutical composition, wherein the pharmaceutical composition comprises the rAAV virion of claim 1.

6. A pharmaceutical composition, wherein the pharmaceutical composition comprises the rAAV virion of claim 2.

7. The pharmaceutical composition according to claim 5, wherein the pharmaceutical composition further comprises a pharmaceutically acceptable excipient.

8. The pharmaceutical composition according to claim 6, wherein the pharmaceutical composition further comprises a pharmaceutically acceptable excipient.

9. The pharmaceutical composition according to claim 7, wherein the rAAV virion is the only active ingredient in the pharmaceutical composition.

10. The pharmaceutical composition according to claim 8, wherein the rAAV virion is the only active ingredient in the pharmaceutical composition.

11. Use of the rAAV virion of claim 1 in the preparation of a product for treating hereditary antithrombin deficiency.

12. Use of the rAAV virion of claim 2 in the preparation of a product for treating hereditary antithrombin deficiency.

13. Use of the rAAV virion of the pharmaceutical composition of claim 7 in the preparation of a product for treating hereditary antithrombin deficiency.

14. Use of the rAAV virion of the pharmaceutical composition of claim 8 in the preparation of a product for treating hereditary antithrombin deficiency.

15. Use of the rAAV virion of claim 3 in the preparation of a product for treating hereditary antithrombin deficiency.

16. Use of the rAAV virion of claim 4 in the preparation of a product for treating hereditary antithrombin deficiency.


SEQUENCE LISTING (3 sequences)

SEQ ID NO: 1 - DNA, 1392 bp, synthetic construct
  source 1..1392: mol_type = other DNA
atgtattccaatgtgataggaactgtaacctctggaaaaaggaaggtttatcttttgtccttgctgctcattggcttctgggactgcgtgacctgtcacgggagccctgtggacatctgcacagccaagccgcgggacattcccatgaatcccatgtgcatttaccgctccccggagaagaaggcaactgaggatgagggctcagaacagaagatcccggaggccaccaaccggcgtgtctgggaactgtccaaggccaattcccgctttgctaccactttctatcagcacctggcagattccaagaatgacaatgataacattttcctgtcacccctgagtatctccacggcttttgctatgaccaagctgggtgcctgtaatgacaccctccagcaactgatggaggtatttaagtttgacaccatatctgagaaaacatctgatcagatccacttcttctttgccaaactgaactgccgactctatcgaaaagccaacaaatcctccaagttagtatcagccaatcgcctttttggagacaaatcccttaccttcaatgagacctaccaggacatcagtgagttggtatatggagccaagctccagcccctggacttcaaggaaaatgcagagcaatccagagcggccatcaacaaatgggtgtccaataagaccgaaggccgaatcaccgatgtcattccctcggaagccatcaatgagctcactgttctggtgctggttaacaccatttacttcaagggcctgtggaagtcaaagttcagccctgagaacacaaggaaggaactgttctacaaggctgatggagagtcgtgttcagcatctatgatgtaccaggaaggcaagttccgttatcggcgcgtggctgaaggcacccaggtgcttgagttgcccttcaaaggtgatgacatcaccatggtcctcatcttgcccaagcctgagaagagcctggccaaggtagagaaggaactcaccccagaggtgctgcaagagtggctggatgaattggaggagatgatgctggtggtccacatgccccgcttccgcattgaggacggcttcagtttgaaggagcagctgcaagacatgggccttgtcgatctgttcagccctgaaaagtccaaactcccaggtattgttgcagaaggccgagatgacctctatgtctcagatgcattccataaggcatttcttgaggtaaatgaagaaggcagtgaagcagctgcaagtaccgctgttgtgattgctggccgttcgctaaaccccaacagggtgactttcaaggccaacaggcctttcctggtttttataagagaagttcctctgaacactattatcttcatgggcagagtagccaacccttgtgttaag

SEQ ID NO: 2 - DNA, 45 bp, synthetic construct
  source 1..45: mol_type = other DNA
ggtggcggtggctcgggcggtggtgggtcgggtggcggcggatct

SEQ ID NO: 3 - DNA, 1653 bp, synthetic construct
  source 1..1653: mol_type = other DNA
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa